(19) United States
(12) Patent Application Publication
(10) Pub. No.: US 2006/0263281 A1
(43) Pub. Date: Nov. 23, 2006

(54) SYSTEMS AND METHODS FOR TREATMENT OF VARIOUS ENVIRONMENTS BY APPLICATION OF OZONE AND STEAM

(75) Inventor: Daniel Christopher Dial, Shelton, WA (US)

Correspondence Address: SPECKMAN LAW GROUP PLLC, 1201 THIRD AVENUE, SUITE 330, SEATTLE, WA, 98101, US

(73) Assignee: DIAL DISCOVERIES LLC, Olympia, WA (US)

(21) Appl. No.: 11/419,704

(22) Filed: May 22, 2006

Related U.S. Application Data
(60) Provisional application No. 60/683,222, filed on May 20, 2005.

(51) Int. Cl. A61L 2/07; A61L 2/20

(52) U.S. Cl. 422/292; 422/305

(57) ABSTRACT

A system for treating various environments having microorganisms is provided. The inventive system comprises a gas distributor, a steam source independently connected to the gas distributor, and an ozone/oxygen source independently connected to the gas distributor, wherein a steam component is generated by the steam source and independently conveyed to the gas distributor, and an ozone/oxygen mixture is generated by the ozone/oxygen source and independently conveyed to the gas distributor, whereby the steam component and the ozone/oxygen mixture are independently distributed to treatment areas and are only mixed at the point of application.

### CROSS REFERENCE TO RELATED APPLICATIONS

This application claims priority under 35 U.S.C. § 119(e) to U.S. Provisional Patent Application No. 60/683,222, filed May 20, 2005.

### FIELD OF INVENTION

The present invention relates to systems and methods for treatment of various environments by applying a steam component and an ozone/oxygen component, particularly, environments having microorganisms and aqueous environments having excessive nitrogen and/or low dissolved oxygen levels.

### BACKGROUND OF THE INVENTION

Many systems and methods known in the art provide decontamination, disinfection, and sterilization of objects, surfaces, and other environments having various types of microorganisms such as viruses, bacteria, spores, pathogens, fungi, molds, and the like.

Disinfection generally involves the use of a chemical agent and/or procedure to eliminate or inhibit the activity of pathogenic microorganisms. In general, three levels of disinfection are recognized: high, intermediate, and low. High-level disinfection kills almost all microorganisms, with the exception of high levels of bacterial spores. Intermediate-level disinfection kills mycobacteria, most viruses, and bacteria. Low-level disinfection kills some viruses and bacteria.

Most disinfectants do not necessarily eliminate all microbial forms (e.g., bacterial endospores), can be quite expensive, and are costly for long term, frequent applications. Also, since many disinfectants are rapidly inactivated by organic matter, objects that are to be disinfected usually need to be cleaned thoroughly with warm water and detergent prior to disinfection, which can be time-consuming. In addition, some disinfectants are toxic or damaging to substrate materials and therefore are potentially dangerous and hazardous to humans, animals, and treated objects and surfaces.

Sterilization generally involves the use of a physical or chemical procedure to destroy various forms of microorganisms. Common sterilizing systems and methods include the use of moist heat by steam autoclaving, ethylene oxide gas, dry heat, and chemical vapor. Conventional steam autoclaving has the advantages of providing relatively rapid turnaround time, low cost per cycle, and not requiring the use of toxic chemicals. However, due to moisture and generally high temperature operation, steam autoclaving may degrade instruments and cannot be used with many plastics. Steam autoclaving also requires the use of distilled, deionized water, which is difficult to generate and can be expensive. Other disadvantages of steam autoclaving include: the high-cost of a sterilizing unit, the need for a relatively large area of space to place the sterilizing, and the need for constant maintenance.

Sterilization using ethylene oxide gas (ETO) requires relatively low temperatures and can be quite effective to inactivate or kill microbial cells. ETO's small molecular size also allows penetration into minute openings and porous substances, allowing sterilant into areas that may not normally receive exposure using other methods of sterilization. However, ETO vaporizes at room temperature and is therefore difficult to contain. It is also extremely explosive unless mixed with other gases. In addition to the problems stemming from ETO's molecular structure, its molecular activity can enable it to combine with many other materials (and/or chemicals) to form new compounds. One hazard of employing ETO is that during sterilization it can easily penetrate thin layers of most plastics and virtually all medical devices. Further, an ETO sterilization cycle can take up to two to three hours and a lengthy aeration time must follow each cycle.

Sterilization by dry heat is generally conducted in an oven equipped with forced air circulation. The heat destroys microorganisms by causing irreversible damage to the cellular components. A typical sterilization process with dry heat is performed at high temperatures (e.g. 160° C. for 2 hours). The challenge in dry heat sterilization is to obtain and maintain an even, high temperature distribution among the goods being sterilized. Furthermore, dry heat sterilization is a very time-consuming process and cannot be used with plastics.

Chemical vapor sterilization generally does not cause corrosion and rusting, and objects sterilized using chemical vapor are dry at the end of the sterilization cycle. However, chemical vapor sterilization generally uses toxic chemicals, which entails specialized and rigorous handling and ventilation requirements.

Various systems utilizing steam or ozone to provide decontamination, disinfection, and sterilization are known. Steam is effective for the elimination of some bacteria, but it is not effective in controlling temperature resistant microorganisms such as mold spores. Ozone, on the other hand, is very effective in eliminating many forms of microorganisms, but it is difficult to maintain consistent delivery of ozone to the environments to be treated because ozone is generally unstable.

It is also known that steam is a useful means for delivering ozone. Thus many systems known in the art provide decontamination, disinfection, or sterilization of various environments by applying a combination of steam and ozone. The combination of steam and ozone provided by those systems is generally premixed within the systems before the steam and ozone mixture reaches a surface, for example, to be treated. However, ozone degrades rapidly at high temperatures, and when ozone is premixed or combined with steam within the above mentioned systems, the amount of usable ozone decreases significantly before it reaches the point of use due to the high temperature of the steam.

In aqueous/water environments, such as marine systems and sewage systems, excessive nitrogen content and/or low levels of dissolved oxygen caused by chemical and bio-waste loads are a common problem. For example, nitrates, if allowed to enter bodies of water, can cause the uncontrollable growth of undesirable oxygen-depleting algae, such as brown algae, orange algae, and red algae, thereby resulting in oxygen depletion of the water environments. Various marine life forms in these water environments can then be harmed or even killed. The decay of the oxygen-depleting algae can further deplete the water of oxygen because algae decay generates high levels of nitrates, which can lower the concentrations of dissolved oxygen in water. In addition, the absence or low level of dissolved oxygen allows anaerobic bacteria to flourish, which can increase toxicity in the water to a lethal level. The oxygen content in water environments depends generally on the environments' chemical and/or biological actions. Dissolved oxygen levels in these water environments sometimes decrease or collapse at certain times of the year. Coupled with industrial wastes, the oxygen level of these water environments can decrease overtime and the environments can eventually fail to produce any oxygen. Disinfection is a common method to substantially eliminate microbial populations in various water environments. The disadvantages of disinfection are discussed above.

Thus, it is desirable to provide systems and methods to overcome the aforementioned disadvantages and problems.

### SUMMARY OF THE INVENTION

In one aspect, systems and methods described herein are provided to substantially eliminate various forms of microorganisms by the generation, distribution, and application of a steam component in combination with an ozone/oxygen mixture or component. In order to prevent premixing of the steam component and the ozone/oxygen mixture, the inventive system is constructed and arranged such that the steam and the ozone/oxygen components are kept separately and independently from each other within the system until mixing occurs at or in proximity to the object, surface, setting, or environment to be treated. Shortly after contacting the treatment area, the ozone breaks down into oxygen and the steam loses its heat energy, and both components safely dissipate into the surroundings. The inventive system thus provides a safe, effective, and non-toxic mixing of steam and ozone/oxygen components only at the point-of-application or treatment.

In general, the system comprises at least one steam source, at least one ozone/oxygen source, and at least one gas distributor. The steam source may be, but is not limited to, a steam generator producing dry steam or wet steam, a boiler, and the like. Various types of steam generators or boilers known in the art may be utilized as a steam source to generate a steam component for use with the inventive system.

The ozone/oxygen source may be, but is not limited to, a premixed ozone/oxygen mixture stored in a reservoir, bottle, tank, and the like or an ozone generator. Many types of ozone generators known in the art may be used with the inventive system. One example is an ozone generator comprising charged grids which charge a portion of the substantially pure oxygen and convert it into ozone, which is diffused in substantially pure oxygen.

In embodiments where the ozone/oxygen source is an ozone generator, substantially pure oxygen from a substantially pure oxygen source is conveyed into the ozone generator, wherein the ozone/oxygen component is generated. Substantially pure oxygen for introduction into the ozone generator may be derived from various sources. The oxygen source may include, but is not limited to, atmospheric air, bottled pressurized oxygen, liquefied oxygen, oxygen tanks, filtered oxygen, compressed oxygen, and the like. The oxygen source may be portable and separate from or installed as part of the system. In systems where the substantially pure oxygen is obtained from atmospheric air, substantially pure oxygen for introduction into the inventive system may be generated by a combination of an air compressor and a molecular sieve. The air compressor draws in the atmospheric air, which is compressed and conveyed to the molecular sieve, where substantially pure oxygen is generated and separated from the compressed atmospheric air. The substantially pure oxygen generated from the oxygen source is then conveyed to the ozone generator where a component of ozone/oxygen is generated.

The volume ratio of the steam and ozone/oxygen components generated is variable. The size and capacity of the system can be varied and may be selected to fit different types of applications. The system may be mobile and portable, semi-permanently installed, or permanently installed. The inventive system may further comprise an on/off switch and/or at least one adjustment control feature, such as dial means, to adjust and control the temperature of the system and the amount and rate of the steam and ozone/oxygen components to be generated. The system may also comprise adjustment means, from which the ratio of the steam and ozone/oxygen components may be programmed to provide a constant or variable ratio of the components.

After the steam component and the ozone/oxygen component are generated, the components are conveyed, separately and independently, intermittently or continuously, from each other, by means of, for example, a backing compressor, towards a gas distributor. The steam and ozone/oxygen components are then distributed to the targeted environment to be treated by means of the gas distributor. In general, the distribution of the steam component and the ozone/oxygen component are synchronically related, and in one example, the steam component and the ozone/oxygen component are delivered simultaneously. Alternatively, the components can be delivered in a timed manner to increase the ozone's effectiveness by introducing the ozone/oxygen component slightly ahead of the steam component. Still alternatively, the components can be delivered in a timed manner by introducing the steam component slightly ahead of the ozone/oxygen component.

The type, size, and configuration of gas distributor may be designed in accordance with the requirements of different environments to be treated. The gas distributor may be, but is not limited to, a nozzle, a tube, a hose, an injector, a bubbler, and the like. It will be appreciated that various embodiments of the gas distributor may be configured to fit different types of applications and a plurality of gas distributors may also be employed.

In one embodiment, the gas distributor is effective for use on large surface areas with few obstructions, such as floors, walls, and carpets. This embodiment of the gas distributor comprises at least one compartment for receiving and distributing an ozone/oxygen mixture, at least one compartment for receiving and distributing a steam component, and a separator plate for separating the compartments. The compartments and the separator plate are connected to each other via a plurality of connectors. The separator plate provides separation between the compartments to ensure that the steam component and the ozone/oxygen component remain separate and independent from each other within the inventive system. Both compartments are provided with a plurality of apertures positioned along the body of the compartments, through which the steam component and the ozone/oxygen component are distributed and applied to treatment areas. In addition, each compartment is provided with at least one inlet positioned about a center of the compartment. The inlets are independently, sealably, and removably connected to tubular members carrying the steam and ozone/oxygen components. In this embodiment, one inlet receives the ozone/oxygen mixture or component, while the other inlet receives the steam component. The steam and ozone/oxygen components are simultaneously and independently distributed and applied to treatment areas via the steam apertures and ozone/oxygen apertures, respectively, allowing only point of application mixing of the two components.

In another embodiment, the gas distributor, effective for use in small spaces, corners, edges, crevasses, and the like, comprises an outer heat shield, a steam supply tubular member, an inner insulating tubular member, and an ozone/oxygen supply tubular member. The outer heat shield is positioned external to the tubular members; the steam supply tubular member is positioned intermediate the outer heat shield and the inner insulating tubular member; the inner insulating tubular member is positioned intermediate the steam supply tubular member and the ozone/oxygen supply tubular member; and the ozone/oxygen supply tubular member is positioned innermost. The tubular members are positioned and arranged such that there are open spaces in between each tubular member. Securing means secure the tubular members to a manifold. The manifold comprises a steam receiving opening that receives a tubular member connected to the steam generator in which the steam component is generated. The ozone/oxygen supply tubular member passes through an end of the manifold and receives the ozone/oxygen component.

The above embodiment of the gas distributor may be further provided with a nozzle sealably connected to the ozone/oxygen supply tubular member and to the steam supply tubular member, through which the steam component and the ozone/oxygen component are independently distributed to the treatment areas. The nozzle comprises a plurality of slightly recessed apertures positioned in a center area of its outer face and its perimeter, wherein the ozone/oxygen mixture component is distributed through the central apertures and the steam component is distributed through the perimeter apertures, via a plurality of ozone/oxygen supply veins and steam supply veins, respectively, thereby allowing only point of application mixing of the two components.

In yet another embodiment, the gas distributor is effective for use in or on generally cylindrical shaped spaces, surfaces, and the like. This embodiment of the gas distributor has a generally cylindrical configuration and is provided with an outer tubular member, an insulating tubular member, a steam supply tubular member, and a cylindrically shaped nozzle. The outer tubular member is positioned external to the other tubular members; the insulating tubular member is positioned intermediate the outer tubular member and the steam supply tubular member; and the steam supply tubular member is positioned innermost. Securing means secures first ends of the tubular members to a manifold, with the second ends of the tubular members being secured to the cylindrical nozzle. The manifold is provided with a steam receiving end and an ozone/oxygen receiving end. The steam receiving end is sealably connected to a tubular member from which the steam component is distributed to the manifold, while the ozone/oxygen receiving end is also sealably connected to a tubular member from which the ozone/oxygen component is distributed to the manifold. The tubular members fit sealably to the manifold and are positioned and arranged such that there are open spaces in-between each tubular member.

The cylindrical nozzle comprises an ozone/oxygen outlet and a steam outlet, with both outlets being separated by a separating means. The ozone/oxygen outlet is provided with a plurality of apertures around the perimeter of the outlet, through which the ozone/oxygen component is independently distributed to treatment areas. The steam outlet is sealably connected to the steam supply tubular member and is provided with a plurality of apertures around its perimeter, through which the steam component is independently distributed to treatment areas, allowing only point of application mixing of the two components.

In other embodiments, the gas distributor of the inventive system may have a half-cylindrical configuration, provided with a half-cylindrically shaped nozzle, or a wedge-like configuration, both configurations being capable of distributing a steam component and an ozone/oxygen component independently.

In another aspect, systems and methods are provided for the treatment of various aqueous/water environments having high nitrogen levels and/or low dissolved oxygen levels by applying a mixture of ozone/oxygen. This aspect of the inventive systems and methods can also be used to reestablish existing ecosystems by increasing the level of oxygen in a given body of water. In this aspect, the inventive system generally involves the introduction of a mixture of ozone/oxygen to raise oxygen levels and to reduce the nitrogen content in various water environments by converting the nitrogen to more environmentally-safe compounds, thereby reducing the anaerobic bacterial populations.

In general, the inventive system for treating various aqueous environments is provided with an oxygen source, an ozone/oxygen source such as an ozone generator, and a distribution manifold array. The oxygen source is conveyed into the ozone/oxygen source, where a mixture/component of ozone/oxygen is generated. The ozone/oxygen component is then released, intermittently or continuously, into the targeted aqueous environment to be treated by means of the distribution manifold array. The size of the inventive system can be variable and may be selected to fit the type of application and the size of the aqueous environment to be treated. The system may be mobile and portable, semi-permanently installed, or permanently installed.

Substantially pure oxygen for introduction to the above system may be derived from various sources. In one embodiment, the substantially pure oxygen generated from the oxygen source is conveyed to the ozone generator where a mixture of ozone/oxygen is produced. The ozone/oxygen component is then conveyed towards the distribution manifold array where the ozone/oxygen component is distributed into the aqueous environment to be treated. The type, size, and configuration of the distribution manifold array may be designed in accordance with the requirements of different aqueous environments to be treated. In one embodiment, the distribution manifold array is provided with a plurality of apertures positioned along the body of the manifold array. In another embodiment, the distribution manifold array may be provided as a series of tubular members having a plurality of apertures.

This aspect of the system can also be used in manure pond or sewage environments to reduce the effluent to a less toxic state whereby the ozone/oxygen component promotes aerobic bacteria to convert the effluent to a more stable form of ammonia. In addition, the ozone/oxygen component generated by the inventive system can decrease the level of disease organisms in the manure pond and sewage environments.

In another example, the inventive system can be installed with and applied to spas, swimming pools, and other similar aqueous environments to reduce or substantially eliminate the microbial populations found in the water. The oxygen is conveyed to an ozone generator where a mixture of ozone/oxygen is produced and released continuously or intermittently, via a gas distributor, into the aqueous environment for disinfection.

In yet another aspect, a sterilizing system is provided. The sterilizing system allows the independent distribution of a steam component and an ozone/oxygen component to the system. Thus, the premixing of the steam and the ozone/oxygen components is prevented in this sterilizing system.

The sterilizing system is provided with an enclosed structure or a substantially enclosed structure, at least one steam distributor, and at least one ozone/oxygen distributor. The enclosed structure may be sealed and may include, but is not limited to, a housing, a chamber, a compartment, a cavity, a case, a room, and the like. The steam distributor receives the steam component from a steam inlet, and the ozone/oxygen distributor receives the ozone/oxygen component from an ozone/oxygen inlet. In one embodiment, the steam distributor and the ozone/oxygen distributor is provided as tubular members, and the steam distributor and the ozone/oxygen distributor enter the enclosed structure generally from a rear area of the sterilizing system. The distributors extend longitudinally along at least one side of the interior of the enclosed structure and may be surrounded by a protective sleeve, and the steam inlet and ozone/oxygen inlet are generally positioned about the rear areas of the distributor.

In this embodiment, the inner wall of the enclosed structure is provided with a plurality of steam outlets and ozone/oxygen outlets. The steam outlets and the ozone/oxygen outlets may be positioned along the sides, the top, and/or below the support tray of the sterilizing system. The steam component is independently received from the steam inlet and passes through the steam distributor, wherein the steam component is distributed to the treatment chamber of the sterilizing system via the steam outlet. Similarly, the ozone/oxygen component is independently received from the ozone/oxygen inlet and passes through the ozone/oxygen distributor, wherein the ozone/oxygen component is distributed to the treatment chamber via the ozone/oxygen outlets. The independent and separate configuration of the steam distributor and the ozone/oxygen distributor allows the steam component and ozone/oxygen component to be mixed at the point of application within the sterilizing system, thus reducing degradation of ozone before the ozone/oxygen component reaches the treatment chamber.

Various types of steam sources known in the art may be utilized to generate the steam component for the sterilizing system and many types of ozone/oxygen sources known in the art may be used with sterilizing system to generate the ozone/oxygen component.

### BRIEF DESCRIPTION OF THE DRAWINGS

The present invention will be described in greater detail in the following detailed description, with reference to the accompanying drawings, wherein:

FIG. 1 shows an exemplary system for treatment of various environments having microorganisms, in which a steam component and an ozone/oxygen component are independently generated and distributed;

FIG. 2A shows a front view of an embodiment of a gas distributor that may be used in connection with the inventive system of FIG. 1;

FIG. 2B shows an exploded side view of the gas distributor of FIG. 2A;

FIG. 2C shows an assembled side view of the gas distributor of FIG. 2A;

FIG. 2D shows a rear view of the gas distributor of FIG. 2A;

FIG. 2E shows a top sectional view of the gas distributor of FIG. 2A, illustrating a compartment to which an ozone/oxygen component is distributed;

FIG. 2F shows another top sectional view of the gas distributor of FIG. 2A, illustrating a compartment to which a steam component is distributed;

FIG. 2G shows a top view of a separator plate of the gas distributor of FIG. 2A;

FIG. 3A shows an assembled side view of another embodiment of a gas distributor of the inventive system;

FIG. 3B shows an exploded side view of the gas distributor of FIG. 3A;

FIG. 4A shows an assembled side view of yet another embodiment of a gas distributor of the inventive system;

FIG. 4B shows an exploded side view of the gas distributor of FIG. 4A;

FIG. 5 shows an exemplary system for treating high nitrogen level and low dissolved oxygen level in various aqueous environments, in which a mixture of ozone/oxygen is delivered to treat the environment;

FIG. 6A shows an exploded external side view of an exemplary sterilizing system in which a steam component and an ozone/oxygen component are independently generated and distributed;

FIG. 6B shows another exploded external view of the sterilizing system of FIG. 6A;

FIG. 6C shows a sectional front view of the sterilizing system of FIG. 6A; and

FIG. 6D shows a sectional side view of the sterilizing system of FIG. 6A.

### DETAILED DESCRIPTION OF THE INVENTION

Treatment of Environments Having Microorganisms

In one aspect, systems and methods described herein are provided for substantially eliminating microorganisms in various environments by generating, distributing, and applying a steam component in combination with a component or mixture of ozone/oxygen to the environment to be treated. The steam component is generally provided at a temperature between about 215° F. and 290° F. (about 100° C. and 145° C.), and the rate of distribution of the steam and ozone/oxygen component is generally between about 2 and 20 liters per minute (1 pm). Since ozone degrades rapidly at high temperatures, in order to maintain the amount of usable ozone at between about 10%-30% at the point of application, the steam component and the ozone/oxygen component are not mixed or combined until both components exit the system at or near the desired point(s) of application. Thus, the steam component and the ozone/oxygen component are generated, distributed, and applied separately and independently from each other. Shortly after contacting the treatment area, the ozone converts into oxygen and the steam loses its heat energy, and both components safely dissipate into the surroundings. This aspect of the inventive systems and methods thus provides a safe, effective, and non-toxic system for disinfection, sterilization, and decontamination by independently generating and distributing a steam component and an ozone/oxygen component within the system and only allows mixing of the two components at points-of-application.

Ozone (O₃) is a molecule that consists of three oxygen atoms, with a delta negative and a delta positive electric charge, and is known to be a molecule that can effectively eliminate various types of microorganisms. The ozone molecule is very unstable and has a short half-life; therefore, it will decay after some time and convert into its original form, oxygen (O₂), according to the reaction presented below:
2O₃≈3O₂

Generally, ozone operates according to the principle of oxidation. During oxidation, the extra oxygen atom is released from the ozone molecule and binds with other materials, leaving oxygen as the resultant molecule. Via oxidation, ozone can act as a disinfectant, sterilant or decontaminant, by oxidizing with various kinds of materials, particularly microorganisms such as viruses, bacteria, spores, pathogens, fungi, molds, and the like. Some additional examples of chemicals that can be oxidized by ozone include, but are not limited to, absorbable organic halogens, nitrite, iron, manganese, cyanide, pesticides, nitrogen oxides, odorous substances, chlorinated hydrocarbons, polychlorinated biphenyls (PCB), and the like.

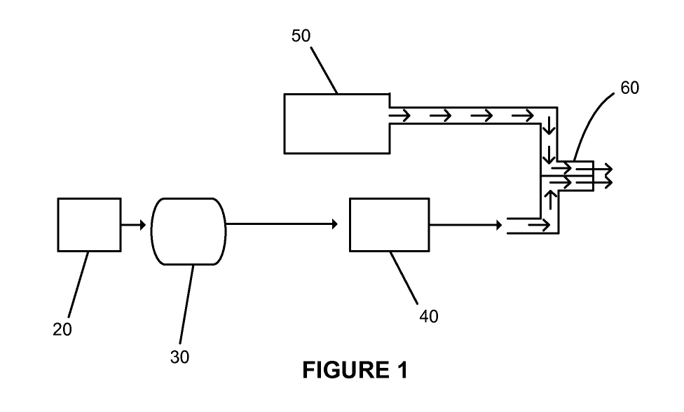

In general, the inventive system 10, as shown in FIG. 1, comprises a plurality of system components that include at least one ozone/oxygen source 40, at least one steam source 50, and at least one gas distributor 60. The system components are in fluid communication with each other, and/or are interconnected to one another, by means of tubular connecting members known in the art. Examples of tubular connecting members include, but are not limited to, oxygen hoses, steam rated hoses, and the like. Ozone/oxygen source 40 and steam source 50 independently provide an ozone/oxygen component and a steam component, respectively, for ultimate supply to gas distributor 60.

The size of inventive system 10 may be variable and ozone/oxygen 40, steam source 50, gas distributor 60, and other components of inventive system 10 may be connected and configured in various ways to fit the desired type of application. Inventive system 10 may be mobile and portable, semi-permanently installed, or permanently installed. Configurations of inventive system 10 include, but are not limited to, rollable models, wearable backpack-like models, and hand-held models. For example, inventive system 10 may be provided with a plurality of wheels or slidable elements. Inventive system 10 can also be installed as part of a vehicle.

Various types of steam generators or boilers known in the art may be utilized as steam source 50 to generate steam for use with inventive system 10. Steam source 50 may be, but is not limited to, a steam generator that produces dry steam, a steam generator that produces wet steam, a boiler, and the like.

Ozone/oxygen source 40 may be, but is not limited to, a premixed ozone/oxygen mixture stored in a reservoir, bottle, tank, and the like or an ozone generator. The ozone generator may have a capacity of at least 90 or 100 grams per hour, 110 or 120 grams per hour, or at least 135 grams per hour. In terms of flow rate and concentration, the capacity should be at least 10 liters per minute at 12%, 13%, 14%, 15%, or higher concentration by weight. Many types of ozone generators known in the art may be used with the inventive system 10. For example, an ozone generator may comprise charged grids which charge a portion of the substantially pure oxygen and convert it into ozone, which is then diffused in the substantially pure oxygen. The charged grids may be constructed with at least two sets of plates, insulated by glass, and charged with a voltage of about 7,500 to about 15,000 volts. The amount of ozone generated may be adjustable by means of, for example, a voltage regulator, to produce about 5% to about 60% ozone in the ozone/oxygen component. In another example, ozone/oxygen source 40 may be a high capacity ozone generator, such as the ASTeX 8403 Ozone Generator, manufactured by Applied Science and Technology, Inc., Woburn, Mass., U.S.A. The ASTeX 8403 has an ozone production rating of 160 grams per hour. At this rate a flow of approximately 12 liters/minute and having a concentration of 19% ozone, by weight, can be supported. Another example of a suitable high capacity ozone generator is the Sumitomo GR-RL Ozone Generator, manufactured by Sumitomo Precision Products Co., Ltd., Hyogo, Japan which has an ozone production rating of 180 g/hr.

The amount of ozone present in the generated ozone/oxygen component/mixture is variable. In one embodiment, the ozone/oxygen component generated by the system is made up of about 5% ozone. In another embodiment, the ozone/oxygen component is made up of about 25% ozone. In yet another embodiment, the ozone/oxygen component is made up of about 40% ozone. In still another embodiment, the ozone/oxygen component is made up of about 50% ozone. Alternatively, the ozone/oxygen component is made up of about 60% ozone.

In embodiments where ozone/oxygen source 40 is an ozone generator, system 10 may be provided with an oxygen separator 30, as needed, to generate substantially pure oxygen from an oxygen source 20. Oxygen for introduction to inventive system 10 may be derived from various sources. It will be appreciated that various oxygen sources 20 known in the art may be employed in the systems and methods of the systems described herein. Oxygen source 20 may include, but is not limited to, atmospheric air, bottled pressurized oxygen, liquefied oxygen, oxygen tanks, filtered oxygen, compressed oxygen, electrolysis supply, and the like. In embodiments of inventive system 10 where oxygen source 20 is atmospheric air, substantially pure oxygen for introduction into the inventive system 10 is generated by an optional oxygen separator 30. Oxygen separator 30 is generally provided with an air compressor and a molecular sieve. Any air compressor known in the art may be used. The air compressor draws in the atmospheric air, which is compressed and conveyed to the molecular sieve, where substantially pure oxygen is generated and separated from the compressed atmospheric air. In one embodiment, the substantially pure oxygen is at least 50% pure. In another embodiment, the substantially pure oxygen is at least 75% pure. In yet another embodiment, the substantially pure oxygen is at least 85% pure. In still another embodiment, the substantially pure oxygen is at least 95% pure.

In this embodiment, the substantially pure oxygen generated by oxygen separator 30 or conveyed from oxygen source 20 is next conveyed to ozone generator 40 where an ozone/oxygen component is generated. The steam component and ozone/oxygen component are then conveyed to gas distributor 60 separately and independently from each other, by means of a first and a second fluid line, where the first fluid line distributes the steam component and the second fluid line distributes the ozone/oxygen component. Both components are then distributed, intermittently or continuously, by means of gas distributor 60, to the targeted environment to be treated.

The volume ratio of the generated steam component and ozone/oxygen component is variable. In one embodiment, the volume ratio of the generated steam component is about 10-90%. In another embodiment, the volume ratio of the generated steam component is about 20-80%. In yet another embodiment, the volume ratio of the generated steam component is about 30-70%. In still another embodiment, the volume ratio of the generated steam component is at least 30%. In an alternative embodiment, the volume ratio of the generated steam component is at least 40%. In another alternative embodiment, the volume ratio of the generated steam component is about 60% and the ozone/oxygen component is about 40%.

After the steam component and ozone/oxygen component are generated, the components are conveyed, by means of, for example, a backing compressor, towards a gas distributor 60. Any backing compressor known in the art may used with the inventive system 10. In general, the distribution of the steam component and the ozone/oxygen component are synchronically related, and in one example, the steam component and the ozone/oxygen component are delivered simultaneously. Alternatively, the components can be delivered in a timed manner to increase the ozone's effectiveness by introducing the ozone/oxygen component slightly ahead of the steam component. Still alternatively, the components can be delivered in a timed manner by introducing the steam component slightly ahead of the ozone/oxygen component.

The type, size, and configuration of gas distributor 60 may be designed in accordance with the requirements of different environments to be treated. Gas distributor 60 may be, but is not limited to, a nozzle, a tube, a hose, an injector, a bubbler, or the like. It will be appreciated that various embodiments of gas distributor 60 may be utilized with inventive system 10. Thus, inventive system 10 may be configured to accommodate and be provided with a plurality of gas distributors 60.

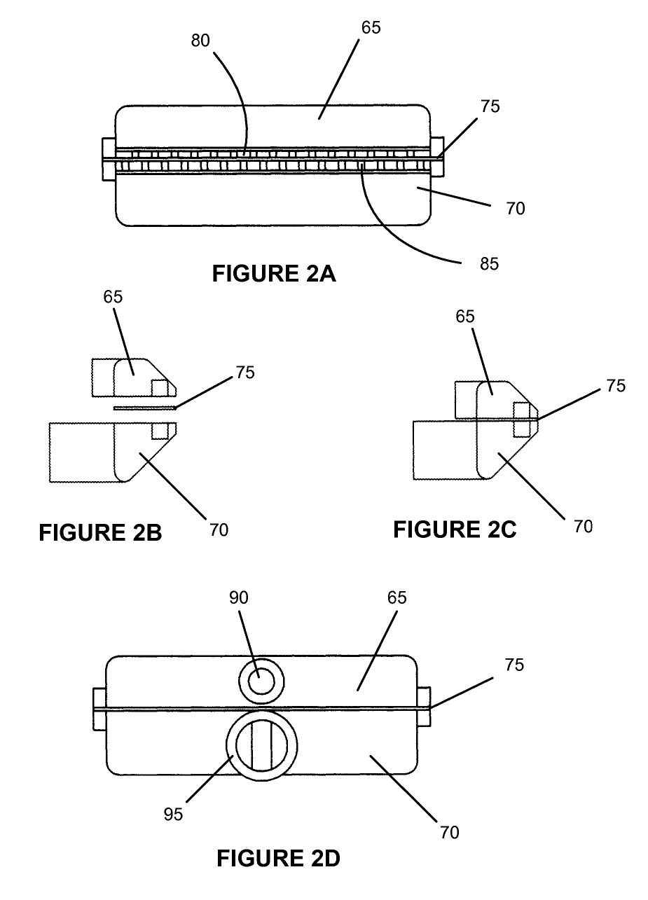

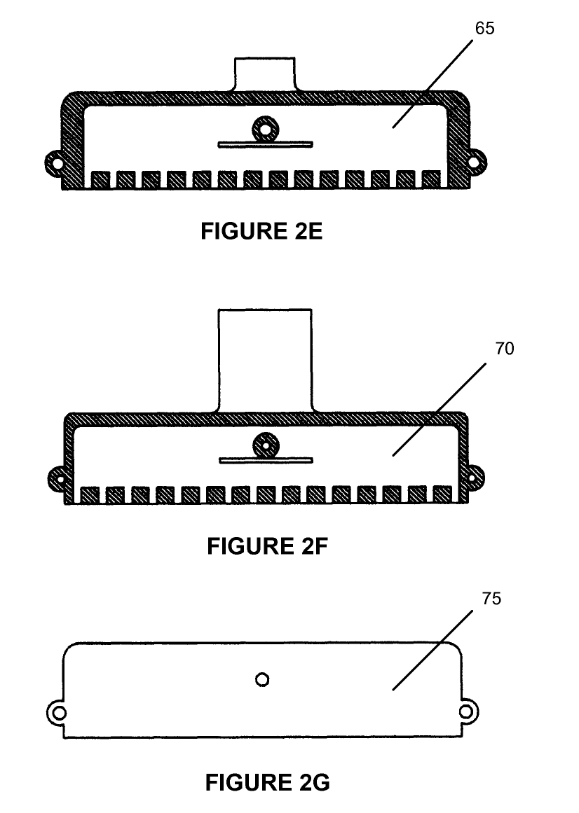

Gas distributor 60 may be provided with a plurality of compartments or tubular members from which, or via which, the steam component and the ozone/oxygen component are distributed and applied to an area to be treated. FIGS. 2A-2G show various views of one embodiment of gas distributor 60. This embodiment of the gas distributor 60 is effective for use on large surfaces with a few obstructions, such as floors, walls and carpets. As shown in FIGS. 2A-2C, this embodiment of gas distributor 60 comprises at least one compartment 65 for receiving and distributing an ozone/oxygen component, at least one compartment 70 for receiving and distributing a steam component, and a separator plate 75 for separating compartments 65 and 70. Compartments 65 and 70 and separator plate 75, which have generally quadrilateral configurations, are connected to each other via a plurality of screws, connectors, and the like, positioned generally around the perimeter of compartments 65 and 70 and plate 75. In one example, the width of this embodiment of the gas distributor is about twenty four inches, but the width is not limited as it may vary in accordance with different types of applications. Separator plate 75 serves to provide separation and sealing between compartments 65 and 70 thereby ensuring that the steam component and the ozone/oxygen component remain separate and independent from each other within inventive system 10. Compartments 65 and 70 are both provided with a plurality of apertures 80 and 85 positioned along the body of compartments 65 and 70, respectively, through which the steam and the ozone/oxygen components are distributed and applied to treatment areas. The size and spacing of the plurality of apertures 80 and 85 may be configured and adjusted to provide various desired levels of the steam and the ozone/oxygen components to a treatment area. As shown in FIG. 2D, compartments 65 and 70 are both provided with at least one inlet 90 and 95 positioned about a center of compartments 65 and 70. Inlets 90 and 95 are independently, sealably, and removably connected to tubular members carrying the steam and ozone/oxygen components. In this embodiment, inlet 90 receives one end of a first tubular member, with the other end of the first tubular member being connected to ozone generator 40, while inlet 95 receives one end of a second tubular member, with the other end of the second tubular member being connected to steam generator 50.

In operation, the ozone/oxygen component generated by ozone generator 40 is conveyed to ozone/oxygen compartment 65 through ozone/oxygen inlet 90, and the steam component generated by steam generator 50 is conveyed to steam compartment 70 through steam inlet 95. The steam and ozone/oxygen components are then independently distributed and applied to treatment areas via steam apertures 85 and ozone/oxygen apertures 80, respectively, allowing point of application mixing of the two components.

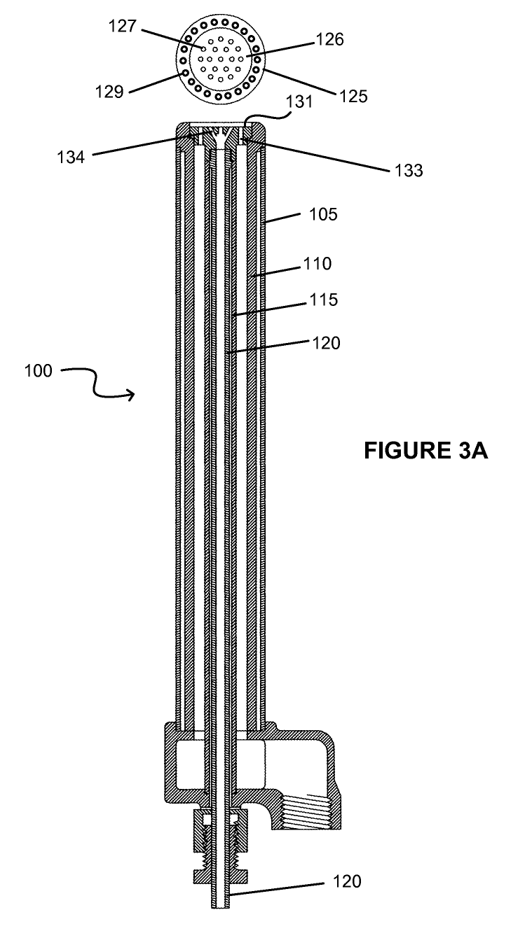

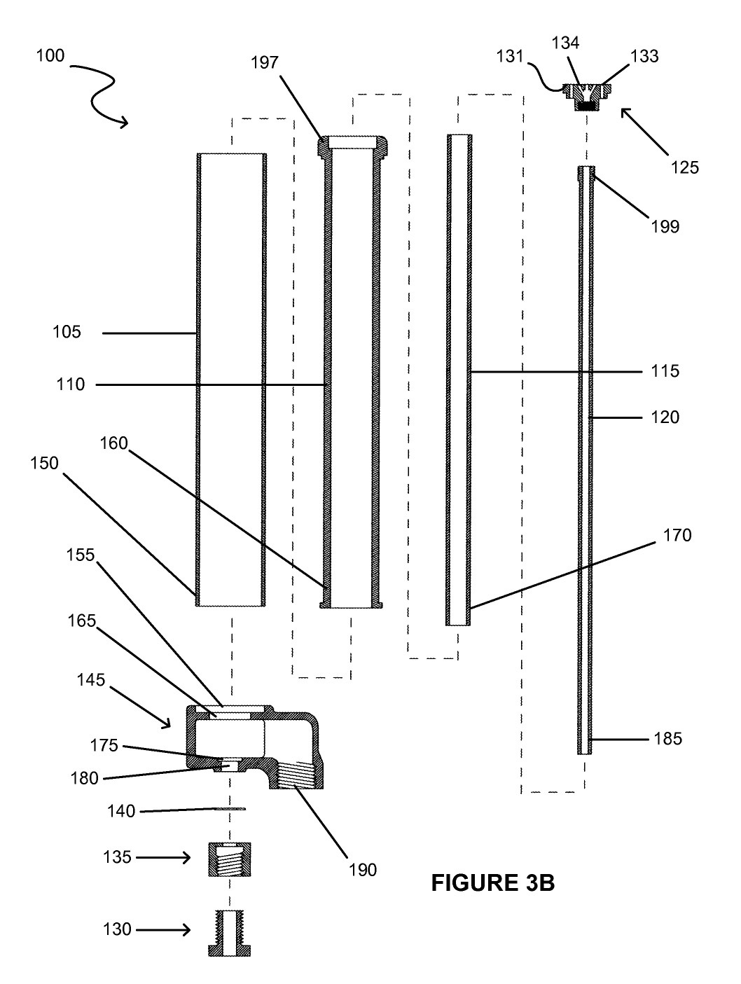

FIGS. 3A and 3B show another embodiment of gas distributor 100 of inventive system 10. This embodiment of gas distributor 100 is effective for use in small spaces, corners, edges, crevasses, and the like. The length and size of gas distributor 100 can be varied to accommodate differing applications. In this embodiment, gas distributor 100 comprises a plurality of nested spaces and elongated tubular members each having a first and a second end. These elongated tubular members are generally insulated to reduce thermal transfer and are defined by an outer heat shield 105, a steam supply tubular member 110, an inner insulating tubular member 115, and an ozone/oxygen supply tubular member 120. As shown in FIG. 3A, when gas distributor 100 is installed, outer heat shield 105 is positioned external to the other tubular members; steam supply tubular member 110 is positioned intermediate outer heat shield 105 and inner insulating tubular member 115; inner insulating tubular member 115 is positioned intermediate steam supply tubular member 110 and ozone/oxygen supply tubular member 120; and ozone/oxygen supply tubular member 120 is positioned innermost. The tubular members are positioned and arranged such that there are open spaces between each tubular member. Generally, the open spaces measures between about 1/64 inch and ½ inch. As shown in FIG. 3B, securing means, including a stem clamp 130, a clamp nut 135, a washer means 140, and a manifold 145, secure the tubular members at first ends of the tubular members to manifold 145. Manifold 145 comprises a steam receiving opening 190 having securing means sealably connected to an end of a tubular member (not shown), with the second of this tubular member being connected to steam generator 50. As shown in FIG. 3A, distal end 150 of outer heat shield 105 fits sealably to receiving gap 155 of manifold 145; distal end 160 of steam supply tubular member 110 is sealably connected to manifold 145 at opening 165; distal area 170 of inner insulating tubular member 115 passes through receiving gap 155 and opening 165, and is sealably connected to manifold 145 at opening 175; and ozone/oxygen supply tubular member 120 passes through receiving gap 155, openings 165, 175 and 180. Ozone/oxygen supply tubular member 120 also passes through and is secured to manifold 145 by stem clamp 130, clamp nut 135, and washer means 140 at its distal end 185. Distal end 185 is sealably connected to an end of a tubular member that is connected to an ozone/oxygen exit area of ozone generator 40.

Gas distributor 100 is further provided with nozzle 125 through which the steam and ozone/oxygen components are independently distributed to treatment areas. In one example, the diameter of the nozzle is between three and four inches, but the size of nozzle 125 is not limited as it may vary in accordance with the types of applications. Nozzle 125 is sealably connected to ozone/oxygen supply tubular member 120 at its proximal end 199 via securing means. Nozzle 125 is also sealably connected to steam supply tubular member 110 at its proximal end 197 via securing means. On its outer face 126, nozzle 125 comprises a plurality of slightly recessed apertures 127 positioned in a center area and a plurality of apertures 129 positioned around the perimeter of the nozzle. The ozone/oxygen component is distributed through apertures 127 and the steam component is distributed through apertures 129. As shown in FIG. 3B, on its inner face 131, nozzle 125 further comprises a plurality of steam supply veins 133 and ozone/oxygen supply veins 134. Steam supply veins 133 are sealably and independently connected to perimeter apertures 129 of nozzle 125, and ozone/oxygen supply veins 134 are sealably and independently connected to central apertures 127.

In operation, since steam supply tubular member 110 and ozone/oxygen supply tubular member 120 are sealably separate from each other, the steam component and ozone/oxygen component are independently distributed to targeted treatment areas via gas distributor 100. The steam component received by manifold 145 travels from steam receiving opening 190 towards opening 165 and receiving gap 155, and through and from distal end 160 of steam supply tubular member 110 to proximal end 197 of tubular member 110. Ozone/oxygen component generated by ozone generator 40 travels from distal end 185 of ozone/oxygen supply tubular member 120 to proximal end 190 of tubular member 120. Through steam supply veins 133 and perimeter apertures 129 of nozzle 125, steam component is independently distributed to the treatment area, and through ozone/oxygen supply veins 134 and central apertures 127, ozone/oxygen component is independently distributed to the treatment area, allowing point of application mixing of the two components.

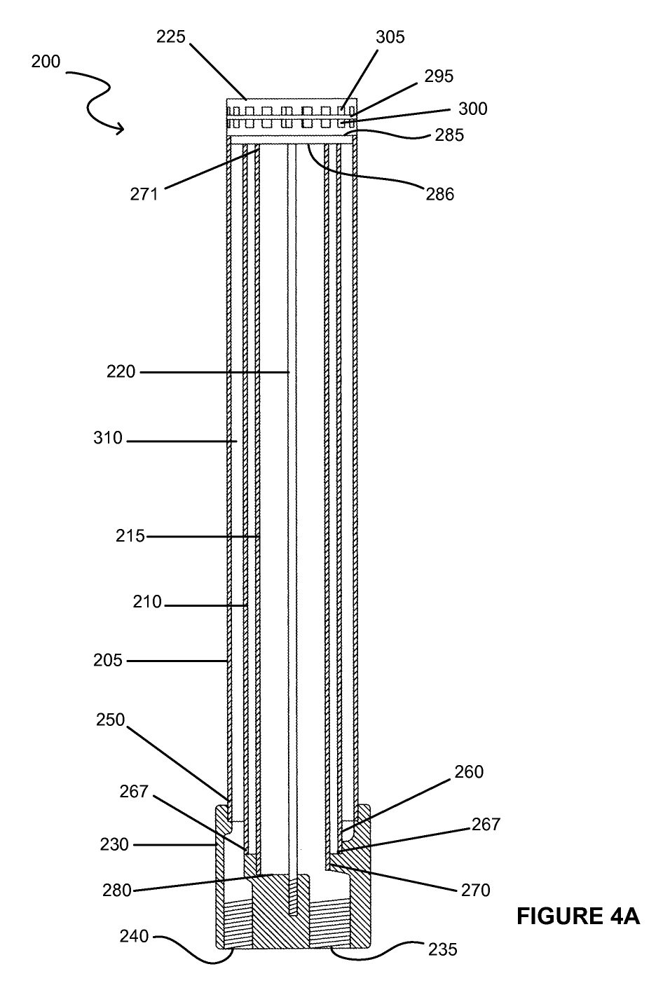

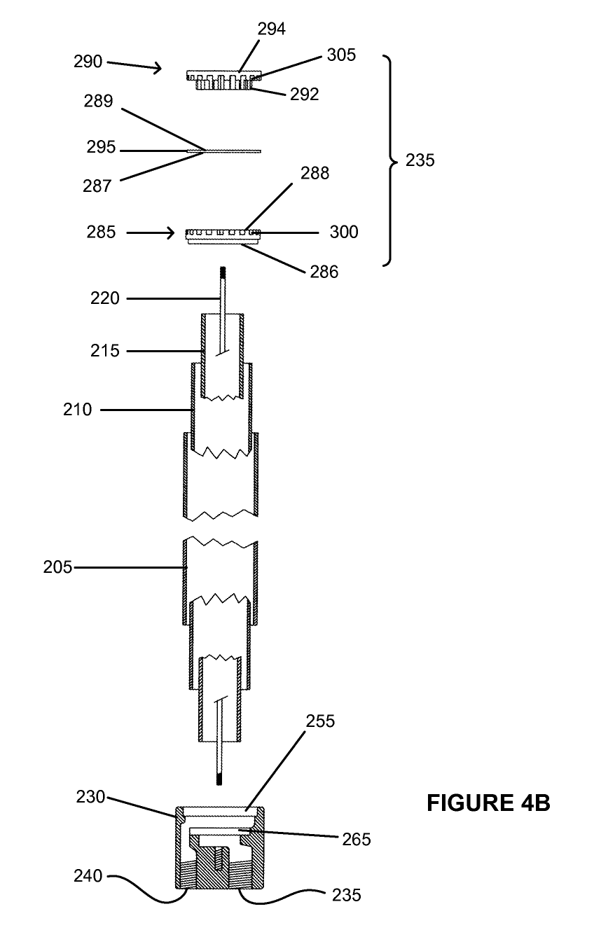

FIGS. 4A and 4B show another embodiment of gas distributor 200 of inventive system 10. This embodiment of gas distributor 200 is effective for use in generally cylindrical shaped spaces, surfaces, and the like. Gas distributor 200 generally has a cylindrical configuration and its length and size can be varied to accommodate differing applications. Gas distributor 200 is provided with a cylindrically shaped nozzle 225. Through cylindrically shaped nozzle 225, the steam component and ozone/oxygen component are distributed in a radial pattern towards the treatment areas. In this embodiment, gas distributor 200 comprises a plurality of nested spaced elongated tubular members having first and second ends. These elongated tubular members are generally insulated to reduce thermal transfer and are defined by an outer tubular member 205, an insulating tubular member 210, and a steam supply tubular member 215. An ozone/oxygen path 310 is defined by the open space formed between outer tubular member 205 and insulating tubular member 210. As shown in FIG. 4A, when gas distributor 200 is installed, outer tubular member 205 is positioned external the other tubular members; insulating tubular member 210 is positioned intermediate outer tubular member 205 and steam supply tubular member 215; and steam supply tubular member 215 is positioned innermost. Securing means 220, such as a stud, secures first ends of the tubular members to a manifold 230, and second ends of the tubular members are secured to nozzle 225. Manifold 230 is provided with a steam receiving end 235 and an ozone/oxygen receiving end 240. Steam receiving end 235 has securing means and is sealably connected to a tubular member from which the steam component generated by steam generator 50 is distributed to manifold 230, while ozone/oxygen receiving end 240 also has securing means and is sealably connected to a tubular member from which the ozone/oxygen component generated by ozone generator 40 is distributed to manifold 230.

As shown in FIG. 4A, distal end 250 of outer tubular member 205 fits sealably to outer receiving gap 255 of manifold 230; distal end 260 of insulating tubular member 210 passes through outer receiving gap 255 of manifold 230 and is sealably connected at notches 267 of inner receiving gap 265; and distal end 270 of steam supply tubular member 215 passes through outer receiving gap 255 of manifold 230 and is sealably connected to manifold 230 at opening 280. The tubular members are positioned and arranged such that there are open spaces in between each tubular member. Generally, the open spaces measures between about 1/64 inch and ⅛ inch.

As shown in FIG. 4B, nozzle 225 of gas distributor 200 has generally a cylindrical configuration and comprises an ozone/oxygen outlet 285 and a steam outlet 290, separated by a separating means 295. Separating means 295 may be, for example, a washer. Ozone/oxygen outlet 285 is provided with a recessed inner face 286 which is sealably connected to a second end of the ozone/oxygen path 310 of gas distributor 200. A perimeter of an outer face 288 of outlet 285 is sealably connected to a perimeter of an inner side 287 of separating means 295 and a plurality of apertures 300 are provided around the perimeter of outer face 288 of outlet 285, through which the ozone/oxygen component is independently distributed to treatment areas. Steam outlet 290 is provided with a recessed and protruding inner face 292. Inner face 292 passes through an outer side 289 of separating means 295 and is sealably connected to a second end of steam supply tubular member 215. Steam outlet 290 is provided with a plurality of apertures 305 around the perimeter of outer face 294 of steam outlet 290, through which the steam component is independently distributed to treatment areas.

In operation, since inner face 292 is sealably connected only to steam supply tubular member 215, the steam and ozone/oxygen components are independently distributed to targeted treatment areas via gas distributor 200. The steam component, generated by steam generator 50, is received by manifold 230 and travels from steam receiving end 235 towards and through distal end 270 of steam supply tubular member 215. The steam component is received at a proximal end 271 of steam supply tubular member 215 and independently exits through apertures 305 of nozzle 225 in a radial pattern. The ozone/oxygen component, generated by ozone generator 40, is received by manifold 230 and travels from ozone/oxygen receiving end 240, through ozone/oxygen path 310 defined by the open space formed between outer tubular member 205 and insulating tubular member 210. The ozone/oxygen component then independently exits through apertures 300 of nozzle 225 in a radial pattern. The independent and separate distribution of the steam and ozone/oxygen components via this embodiment of gas distributor 200, prevents mixing of the two components before they reach the point of application.

In still another embodiment, the gas distributor of the inventive system has a half-cylindrical configuration and is provided with a half-cylindrically shaped nozzle capable of distributing a steam component and ozone/oxygen component independently. The half-cylindrical gas distributor is also effective for use in confined spaces and surfaces with limited access.

In an alternative embodiment, the gas distributor of the inventive system has a wedge-like configuration and is provided with a nozzle capable of distributing steam and ozone/oxygen components independently. The wedge-like gas distributor is effective for use in corners and other wedge-like spaces.

The inventive system may further comprise an on/off switch and/or at least one adjustment control feature, such as dial means, to adjust and control the temperature of the system and the amount and rate of the steam and ozone/oxygen components to be generated. The system may also comprise adjustment means, from which the ratio of the steam and ozone/oxygen components may be programmed to provide a constant or variable ratio of the components.

Treatment of Aqueous Environments Having Excessive Nitrogen and/or Low Dissolved Oxygen Levels

In another aspect, systems and methods are provided for treating various aqueous/water environments having high nitrogen levels and/or low dissolved oxygen levels. The systems and methods generally involve the introduction of a mixture of ozone/oxygen to raise oxygen levels and to reduce the nitrogen content in various water environments by converting the nitrogen to more environmentally-safe compounds, thereby reducing the anaerobic bacterial populations. The inventive system requires little maintenance, reduces the need for an elaborate plumbing system, and is operable without requiring excessive energy sources.

Generally, when ozone is added to a water environment, it reacts with the nitrogen present in the water by donating an extra oxygen atom from the ozone to the nitrogen molecules, or nitrate chains, suspended in the water. The additional oxygen atom causes the larger nitrate compounds to attract other nitrates present in the water environment. The nitrate compounds tend to aggregate and eventually sink to the bottom of the water environment. This process reduces the available nitrogen in the water environment needed for the growth of oxygen-depleting algae and anaerobic bacteria. Thus, the oxygen-depleting algae and anaerobic bacteria eventually die and are replaced by the more normal aerobic bacteria, and green and purple algae.

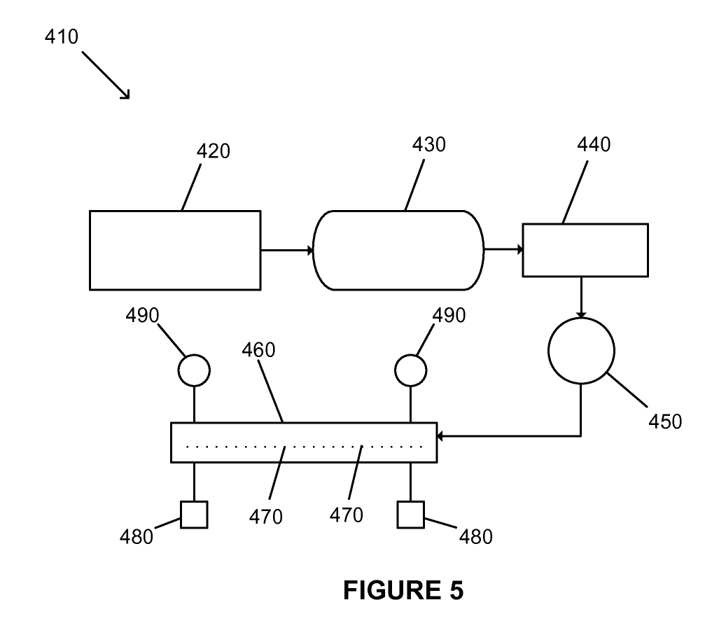

FIG. 5 shows an exemplary system 410 of the present invention that can be used to treat various large bodies of aqueous/water environments having high nitrogen levels and/or low dissolved oxygen levels for water remediation purposes. The large bodies of water environments may include, but are not limited to, such as lakes, rivers, canals, large ponds, and the like. In this embodiment, the inventive system 410 is provided with an oxygen source comprising an air compressor 420 and a molecular sieve 430 to facilitate the introduction of an ozone/oxygen component into the water environment to be treated, an ozone/oxygen source 440 such as an ozone generator, and a distribution manifold array 460. The system 410 may be mobile and portable, semi-permanently installed, or permanently installed about the targeted water environment to be treated.

In the exemplary embodiment shown in FIG. 5, the oxygen source comprises an air compressor 420 and a molecular sieve 430. Any air compressor known in the art may be used. Air compressor 420 draws in atmospheric air, which is compressed and conveyed to molecular sieve 430, where substantially pure oxygen is generated and separated from the compressed atmospheric air. In one embodiment, the substantially pure oxygen is at least 50% pure. In another embodiment, the substantially pure oxygen is at least 75% pure. In yet another embodiment, the substantially pure oxygen is at least 85% pure. In still another embodiment, the substantially pure oxygen is at least 95% pure.

The substantially pure oxygen is next conveyed to ozone generator 440 where a mixture of ozone/oxygen is produced. The amount of ozone present in the generated ozone/oxygen component/mixture is variable. In one embodiment, the ozone/oxygen component generated by the system is made up of about 5% ozone. In another embodiment, the ozone/oxygen component is made up of about 25% ozone. In yet another embodiment, the ozone/oxygen component is made up of about 40% ozone. In still another embodiment, the ozone/oxygen component is made up of about 50% ozone. Alternatively, the ozone/oxygen component is made up of about 60% ozone.

The ozone/oxygen component is then conveyed by means of a backing compressor 450 towards a gas distributor 460 where the ozone/oxygen component is distributed, intermittently or continuously, into the water environment to be treated. Any backing compressor may used with the inventive system. The type, size, and configuration of gas distributor may be designed in accordance with the requirements of different water environments to be treated. The gas distributor is generally constructed from materials that are non-corrosive and non-reactive in water environments. In the embodiment shown in FIG. 5, the gas distributor is a distribution manifold array 460, which is provided with a plurality of apertures 470 positioned along its body. The size and spacing of the plurality of apertures 470 may be configured and adjusted to provide various desired levels of the ozone/oxygen component to be distributed to a water environment to be treated.

In another embodiment, the gas distributor may be constructed as a series of tubular members having a plurality of apertures positioned along the body of the tubular members. The length of the tubular members forming this example of the gas distributor is generally substantially greater than the diameter of the tubes. The tubular members may be configured in a linear or curved arrangement, and may be provided in various configurations including, but not limited to, circular, polygonal, and the like. In other embodiments, the gas distributor may be, but is not limited to, a tube, a nozzle, a hose, an injector, a bubbler, or the like.

The distribution manifold array 460 is generally suspended in the body of water environment to be treated. Distribution manifold array 460 may also be positioned at a certain stratification layer (a clear boundary of layers from top to bottom in a body of waters that rarely mix with each other). For example, distribution manifold array 460 may be positioned near the bottom edge of a stratification layer so that the ozone/oxygen component can be dispersed thoroughly. In one example, in order to provide effective distribution of the diffused ozone/oxygen component in a body of water environment, distribution manifold array 460 of the inventive system 410 is positioned perpendicular to the tidal flow of the water in order to allow circulation of a large volume of tidal water around the distribution manifold array 460. To prevent an excessive ozone/oxygen supply by the inventive system during the periods of slack tides, the inventive system may be paused and restarted at the beginning of the next tidal cycle.

To prevent unwanted disturbance of sediments at the bottom of the water environment, distribution manifold array 460 is generally mounted or suspended at least about 1-2 feet above the bottom of the body of water to be treated and about thirty to about sixty feet below the air/water interface surface. As illustrated in FIG. 5, distribution manifold array 460 may be suspended in the water by means of at least one ballast component 480 mounted to the manifold array 460 in combination with at least one floatation device 490, which is also mounted to the manifold array 460.

As distribution manifold array 460 is deployed into a water environment to be treated, the ballast components 480 facilitate placement of the system 410 beneath the surface of the water. In one embodiment, at least one ballast component 480 may be provided with a cable leader, measuring generally about twenty to about thirty inches, or another type of sensor, to prevent the manifold array 60 from coming into contact with the bottom of the water.

Floatation device 490 may be provided as a plurality of sealed bladders that are fillable with a buoyant material, such as air, to provided suspension of the distribution manifold array 460 in the water environment at the depth of water desired. The buoyant material may be introduced into one or more sealed bladders, for example, to counter the weight of the ballast components 480. To suspend the distribution manifold array 460 at the desired depth, the number of floatation devices 490 may vary and the amount of buoyant material may be adjusted. In yet another embodiment, one or more floatation devices 490 may be connected to a floating marker. Once the treatment cycle of the water environment is completed or paused, the distribution manifold array 460 may be retrieved from the water environment. Retrieval of the manifold array 460 is generally accomplished by increasing the inflation of the floatation devices 490 to effectively float the distribution manifold array 460 towards the surface of the water environment.

The time required for the remediation process facilitated by the systems and methods of the present invention depends on several factors, such as, but not limited to, the rate of oxygen cycle recovery, nitrogen concentrations, rate of oxygenation, mean water temperature, and the like.

This system can also be used in manure pond environments to reduce the effluent to a less toxic state. In this application, the ozone/oxygen component promotes aerobic bacteria that convert the effluent to a more stable form of ammonia. In addition, the ozone/oxygen component generated by the inventive system can decrease the level of diseased organisms in the manure pond environments. The inventive system can thereby improve the effectiveness of the effluent as a viable fertilizer for field use or disposal without destroying the surrounding environment. In this embodiment, the inventive system may be provided with any oxygen source as described above, an ozone generator, and any gas distributor as described above. The inventive system for use in this application may be mobile and portable, semi-permanently installed, or permanently installed about the targeted manure pond to be treated. The ozone/oxygen component generated in this embodiment may contain about 25%-50% ozone.

In yet another embodiment, this system can be used to treat sewage systems that contain high levels of nitrogen compounds. Such a system can process many times the normal amounts of raw sewage, and the resulting liquids from the process contain low levels of nitrogen and therefore are safe enough to be discharged back into the water system. In this embodiment, the inventive system may be provided with any oxygen source as described above, an ozone generator, and any gas distributor as described above. The inventive system for use in this application may be mobile and portable, semi-permanently installed, or permanently installed about the targeted manure pond to be treated. The ozone/oxygen component generated in this embodiment may contain about 25%-50% ozone.

In still another embodiment of the inventive system, the system can be installed with or applied to spas, swimming pools, whirlpools, small ponds, and other similar water environments to reduce or substantially eliminate the microbial populations found in the water. In this embodiment, the system is generally mobile and portable, but may alternatively be semi-permanently installed, or permanently installed proximal to the water environment to be treated. The substantially pure oxygen content feed may be provided in the form of, but is not limited to, bottled pressurized oxygen, liquefied oxygen, oxygen tanks, filtered oxygen, and the like, whereby the substantially pure oxygen content feed is directly supplied to the system. The substantially pure oxygen is conveyed to an ozone generator where a mixture of ozone/oxygen is produced. The ozone/oxygen component generated in this embodiment preferably contains about 5%-10% ozone. The ozone/oxygen component is next released, continuously or intermittently, via a gas distributor such as a tube, a hose, a bubbler, a nozzle, a manifold, an injector, or the like, into the water environment for disinfection.

For example, an operator may transport the system to a swimming pool, where he/she turns on the system and places the gas distributor, such as a tube or a bubbler, into the swimming pool where the ozone/oxygen component is distributed intermittently or continuously. If the ozone/oxygen component is distributed intermittently, the ozone content is preferred to be about 10-20%. If the ozone/oxygen component is distributed continuously, the ozone content is preferred to be about 5-10%. This system is suitable for application about two to three times per week.

Sterilizing Systems

In yet another aspect, a sterilizing system is provided. The sterilizing system allows the independent distribution of a steam component and an ozone/oxygen component to the system. The inventive sterilizing system substantially eliminates the degradation of ozone by preventing the premixing of the steam and the ozone/oxygen components. The inventive sterilizing system is constructed and arranged such that the steam component and the ozone/oxygen component are kept separately and independently from each other within the system until both components reach the point of application or treatment. The inventive sterilizing system thus provides a safe, effective, and non-toxic mixing of steam and ozone/oxygen components only at the point-of-application. The sterilizing system may be constructed to operate under atmospheric pressure or high pressure.

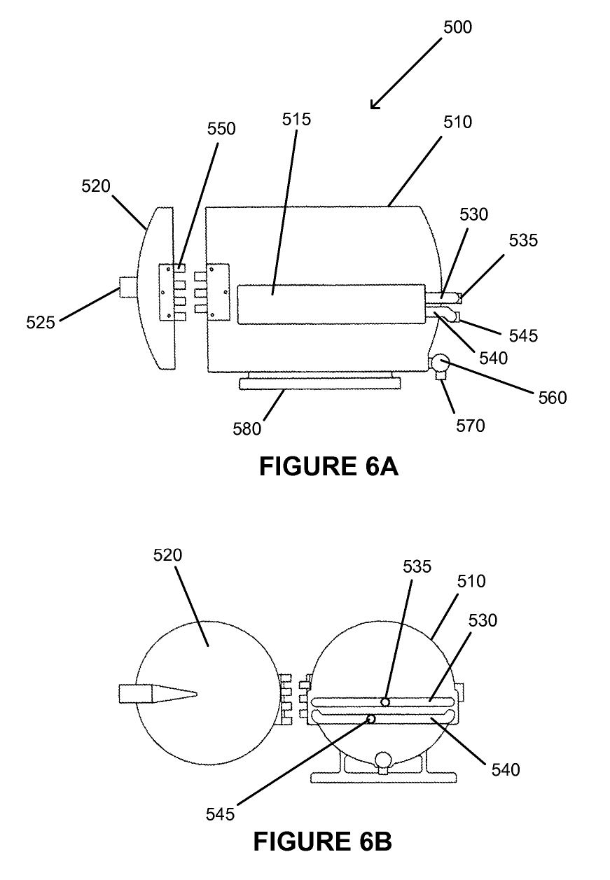

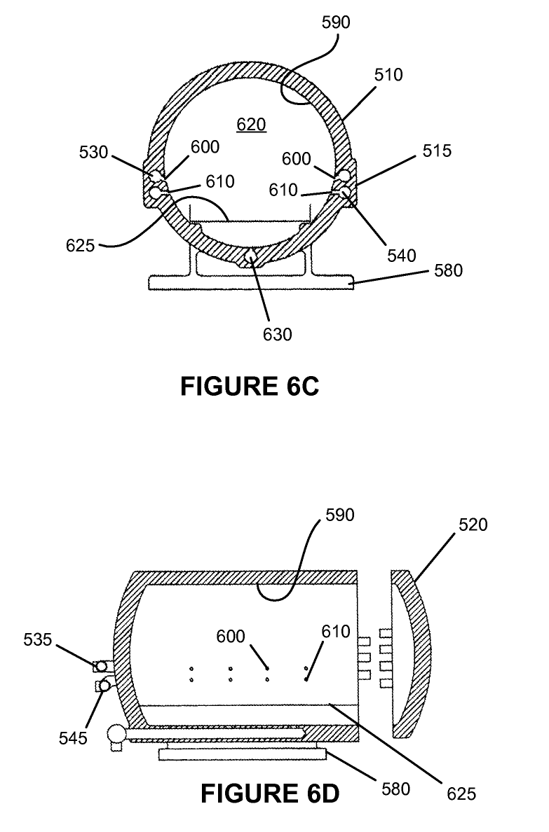

In general, the sterilizing system comprises an enclosed structure, at least one steam distributor, and at least one ozone/oxygen distributor. FIG. 6A shows an external side view of an embodiment of the inventive sterilizing system 500. Sterilizing system 500 is provided with enclosed structure 510, door seal 520, steam distributor 530, and ozone/oxygen distributor 540. Door seal 520 is provided with any latches 525 known in the art and is hingeably connected to enclosed structure 510 via hinging means 550. Steam distributor 530 receives the steam component from a steam inlet 535, and ozone/oxygen distributor 540 receives the ozone/oxygen component from an ozone/oxygen inlet 545. In this embodiment, sterilizing system 500 is additionally provided with a relief valve 560 connected to an exhaust outlet 570, and sterilizing system 500 may be mounted to a mounting base 580.

As shown in FIGS. 6A and 6B, steam distributor 530 and ozone/oxygen distributor 540 are provided as tubular members, although other types of configurations of steam distributor 530 and ozone/oxygen distributor 540 may be utilized. In the embodiment shown in FIGS. 6A and 6B, steam distributor 530 and ozone/oxygen distributor 540 enter enclosed structure 510 from a rear area of sterilizing system 500. Distributors 530 and 540 extend longitudinally along at least one side of enclosed structure 510 and may be enclosed by a protective sleeve 515, which is a part of enclosed structure 510. Distributors 530 and 540 may also extend along other areas of enclosed structure 510. Steam inlet 535 and ozone/oxygen inlet 540 generally enter the rear areas of distributors 530 and 540, although inlets 535 and 545 may enter distributors 503 and 540 at other areas.

As shown in FIGS. 6C and 6D, inner wall 590 of enclosed structure 510 is provided with a plurality of steam outlets 600 and ozone/oxygen outlets 610. Steam outlets 600 and ozone/oxygen outlets 610 may be positioned along the sides, the top, and/or below the support tray 625 of sterilizing system 500. The steam component is independently received from steam inlet 535 and passes through steam distributor 530, wherein the steam component is distributed to treatment chamber 620 via steam outlets 600. Similarly, the ozone/oxygen component is independently received from ozone/oxygen inlet 545 and passes through ozone/oxygen distributor 540, wherein the ozone/oxygen component is distributed to treatment chamber 620 via ozone/oxygen outlets 600. The independent and separate configuration of steam distributor 530 and ozone/oxygen distributor 540 allows the steam component and ozone/oxygen component to be mixed at the point of application within sterilizing system 500, thus reducing degradation of ozone before the ozone/oxygen component reaches treatment chamber 620. Sterilizing system 500 may further provided with a drainage and pressure relief port 630.

Various types of steam sources known in the art, such as steam generators or boilers, may be utilized to generate steam for sterilizing system 500 and steam generators that produce either dry steam or wet steam may be used. Many types of ozone/oxygen sources known in the art, such as premixed ozone/oxygen sources and ozone generators, may be used with sterilizing system 500. One example of an ozone generator comprises charged grids which charge a portion of the substantially pure oxygen and convert it into ozone, which is diffused in the substantially pure oxygen. In another example, a “corona” discharge ozone generator may be utilized. A “corona” discharge ozone generator operates by passing dry and oxygen-containing gas through an electrical field. The electrical charge is diffused over a dielectric surface, creating an electrical field, or “corona”.

The amount of ozone present in the generated ozone/oxygen component/mixture is variable. In one embodiment, the ozone/oxygen component generated by the system is made up of about 5% ozone. In another embodiment, the ozone/oxygen component is made up of about 25% ozone. In yet another embodiment, the ozone/oxygen component is made up of about 40% ozone. In still another embodiment, the ozone/oxygen component is made up of about 50% ozone. Alternatively, the ozone/oxygen component is made up of about 60% ozone.

In general, the steam component and the ozone/oxygen component are synchronically related, and in one example, the steam component and the ozone/oxygen component are delivered simultaneously into treatment chamber 620. Alternatively, the components can be delivered in a timed manner to increase the ozone's effectiveness by introducing the ozone/oxygen component slightly ahead of the steam component during the sterilizing treatment cycle. Still alternatively, the components can be delivered in a timed manner by introducing the steam component slightly ahead of the ozone/oxygen component during the sterilizing treatment cycle.

While certain embodiments of the present invention have been described, it will be understood that various changes may be made in the above constructions without departing from the scope of the invention. It is intended that all matter contained in the above description or shown in the accompanying drawings shall be interpreted as illustrative and not in a limiting sense.

I claim:

1. A system for treating an environment having microorganisms, comprising:
(a) at least one gas distributor;
(b) at least one steam source independently connected to the gas distributor; and
(c) at least one ozone/oxygen source independently connected to the gas distributor, wherein a steam component is generated by the at least one steam source and independently conveyed to the at least one gas distributor, wherein an ozone/oxygen mixture is generated by the at least one ozone/oxygen source and independently conveyed to the at least one gas distributor, whereby the steam component and the ozone/oxygen mixture are independently distributed to a treatment area and the steam component and the ozone/oxygen mixture are only mixed at a point of application.

2. The system of claim 1, wherein the at least one steam source is a steam generator and the at least one ozone/oxygen source is an ozone generator.

3. The system of claim 1, further comprising at least one substantially pure oxygen source, wherein substantially pure oxygen is conveyed from the at least one substantially pure oxygen source to the at least one ozone/oxygen source and an ozone/oxygen component is generated from the at least one ozone/oxygen source.

4. The system of claim 3, wherein the at least one substantially pure oxygen source is selected from a group consisting of: atmospheric air, bottled pressurized oxygen, liquefied oxygen, oxygen tanks, filtered oxygen, compressed oxygen, and mixtures thereof.

5. The system of claim 1, wherein the ozone/oxygen mixture contains at least 25% ozone.

6. The system of claim 1, wherein the ozone/oxygen mixture contains at least 40% ozone.

7. The system of claim 1, wherein the ozone/oxygen mixture contains at least 50% ozone.

8. The system of claim 1, wherein the ozone/oxygen mixture is distributed intermittently from the system.

9. The system of claim 1, wherein the ozone/oxygen mixture is distributed continuously from the system.

10. The system of claim 1, wherein the at least one gas distributor is selected from a group consisting of: distribution manifold arrays, tubes, nozzles, hoses, injectors, bubblers, and combinations thereof.

11. The system of claim 1, wherein the at least one gas distributor comprises:
(a) at least one ozone/oxygen compartment for receiving and distributing the ozone/oxygen component;
(b) at least one steam compartment for receiving and distributing the steam component; and
(c) a separator plate for separating the at least one ozone/oxygen compartment and at least one steam compartment,
wherein the at least one ozone/oxygen compartment and the at least one steam compartment are each provided with at least one inlet positioned about a center of the compartment for independently receiving the ozone/oxygen and steam components, respectively, and wherein each of the at least one ozone/oxygen compartment and the at least one steam compartment is provided with a plurality of apertures positioned along the body of the compartment, through which the steam and ozone/oxygen components are independently distributed and applied to treatment areas, allowing only point of application mixing of the components.

12. The system of claim 1, wherein the at least one gas distributor comprises:
(a) a manifold having a steam receiving end through which the steam component enters the manifold and an ozone/oxygen receiving end through which the ozone/oxygen component enters the manifold;
(b) a nozzle comprising a plurality of central apertures positioned in a center area of the outer face of the nozzle for distribution of the ozone/oxygen component and a plurality of perimeter apertures positioned around the perimeter of the nozzle for distribution of the steam component;
(c) an outer heat shield;
(d) a steam supply tubular member;
(e) an inner insulating tubular member; and
(f) an ozone/oxygen supply tubular member,
wherein
(i) the outer heat shield is positioned external to the steam supply tubular member, the inner insulating tubular member and the ozone/oxygen supply tubular member; the steam supply tubular member is positioned intermediate the outer heat shield and the inner insulating tubular member; the inner insulating tubular member is positioned intermediate the steam supply tubular member and the ozone/oxygen supply tubular member; the ozone/oxygen supply tubular member is positioned innermost; and the heat shield and the tubular members are separated from each other by spaces; and
(ii) the steam supply tubular member and the ozone/oxygen supply tubular member is each separately connected to the manifold at a first end and to the nozzle at a second end.

13. The system of claim 1, wherein the at least one gas distributor comprises:
(a) a manifold having a steam receiving end through which the steam component enters the manifold and an ozone/oxygen receiving end through which the ozone/oxygen component enters the manifold;
(b) a nozzle having an ozone/oxygen outlet provided with a plurality of apertures through which the ozone/oxygen component is distributed to the treatment area and a steam outlet provided with a plurality of apertures through which the steam component is distributed to the treatment area;
(c) an outer tubular member;
(d) an insulating tubular member
(e) a steam supply tubular member,
wherein
(i) the outer tubular member is positioned external to the insulating tubular member and the steam supply tubular member, the insulating tubular member is positioned intermediate the outer tubular member and the steam supply tubular member, the steam supply tubular member is positioned innermost, and the tubular members are separated from each other by spaces,
(ii) each of the tubular members is separately secured to the manifold at a first end and separately secured to the nozzle at a second end,

14. The system of claim 1, wherein the gas distributor has a half-cylindrical configuration.

15. The system of claim 1, wherein the gas distributor has a wedge shaped configuration.

16. A system for treating an aqueous environment having excessive nitrogen levels and/or low dissolved oxygen levels, comprising:
(a) at least one substantially pure oxygen source;
(b) at least one ozone/oxygen source connected to the at least one substantially oxygen source; and
(c) at least one gas distributor connected to the at least one ozone/oxygen source, wherein substantially pure oxygen is conveyed to the at least one ozone/oxygen source, a component of ozone/oxygen is generated in the at least one ozone/oxygen source, and the ozone/oxygen component is conveyed to the at least one gas distributor for distribution into the aqueous environment to be treated.

17. The system of claim 16, wherein at least one gas distributor is selected from the group consisting of: distribution manifold arrays, tubes, nozzles, hoses, injectors, bubblers, and combinations thereof.

18. A sterilizing system, comprising:
(a) an enclosed structure;
(b) at least one steam distributor independently and sealably connected to the enclosed structure; and
(c) at least one ozone/oxygen distributor independently and sealably connected to the enclosed structure,
wherein a steam component is independently conveyed to the at least one steam distributor and an ozone/oxygen component is independently conveyed to the at least one ozone/oxygen distributor, whereby the steam component and the ozone/oxygen component are independently distributed to the enclosed structure and the components are only mixed at a point of application.

19. The sterilizing system of claim 18, wherein the steam distributor independently receives the steam component from a steam inlet, wherein the ozone/oxygen distributor independently receives the ozone/oxygen component from an ozone/oxygen inlet, and wherein the enclosed structure comprises a plurality of steam outlets connected to the steam distributor and a plurality of ozone/oxygen outlets connected to the ozone/oxygen distributor.

20. The sterilizing system of claim 18, wherein the enclosed structure further comprises a door seal sealably and hingeably connected to the enclosed structure, a relief valve connected to an exhaust outlet, and a drainage and pressure relief port.
